(19) United States
(12) Patent Application Publication
(10) Pub. No.: US 2022/0138939 A1
(43) Pub. Date: May 5, 2022

(54) Systems and Methods for Digital Pathology

(71) Applicant: The Regents of the University of California, Oakland, CA (US)

(72) Inventors: Bahram Jalali, Oakland, CA (US); Madhuri Suthar, Oakland, CA (US); Cejo Konuparamban Lonappan, Oakland, CA (US); Antoni Ribas, Oakland, CA (US); Theodore Scott Nowicki, Oakland, CA (US); Jia Ming Chen, Oakland, CA (US)

(73) Assignee: The Regents of the University of California, Oakland, CA (US)

(21) Appl. No.: 17/431,138

(22) PCT Filed: Feb. 14, 2020
(86) PCT No.: PCT/US20/18424

§ 371 Date: Aug. 13, 2021

Related U.S. Application Data
(60) Provisional application No. 62/806,585, filed on Feb. 15, 2019.

(51) Int. Cl. G06T 7/00; G06V 20/69; G06V 10/25; G06V 10/40; G02B 21/36; G16H 10/40; G16H 70/60; G16H 30/40

(52) U.S. Cl. CPC G06T 7/0012; G06V 20/693; G06V 10/25; G06V 10/40; G02B 21/365; G06T 2207/30024; G16H 70/60; G16H 30/40; G06T 2207/10056; G06T 2207/20081; G06T 2207/20084; G16H 10/40

(57) ABSTRACT

Systems and methods for digital pathology in accordance with embodiments of the invention obtain a whole slide image of a microscope slide that includes a registration mark, wherein the registration mark is associated with a coordinate system. The method inputs the whole slide image into a region identification (RI) model to extract features of the whole slide image and generate feature vectors for the extracted features, detects a presence of a region of interest (ROI) based on the feature vectors, determines a set of coordinates of the ROI in the coordinate system, and translates a microscope stage of a microscope holding the microscope slide to a position corresponding to the coordinates of the ROI. The method captures a field of view (FOV) image with the microscope and inputs the FOV image into a grading model to determine a pathology score that indicates a likelihood of a presence of a disease.

### STATEMENT REGARDING FEDERALLY SPONSORED RESEARCH OR DEVELOPMENT

This invention was made with government support under Grant Numbers CA197633 and GM107924, awarded by the National Institutes of Health, and Grant Number N00014-14-1-0505, awarded by the U.S. Navy, Office of Naval Research. The government has certain rights in the invention.

### FIELD OF THE INVENTION

This disclosure relates to image processing and particularly to a digital pathology system that performs feature detection to improve workflow between a whole slide scanner and a pathology microscope.

### BACKGROUND

Digital pathology is an extremely powerful tool in the field of cancer research and clinical practice. Although the availability of advanced whole slide scanners has enabled complete digitization of microscope slides, the development of automated histopathology image analysis tools has been challenging due in part to the large file size of high-resolution digital images captured by whole slide scanners. In some cases, a pathologist may use an image of a microscope slide captured with a whole slide scanner to visually identify pathology features relevant to a potential diagnosis of a patient. However, the pathologist is generally required to manually load the slide into a separate pathology microscope and locate relevant regions of interest (ROIs), in order to perform an in-depth visual inspection of the ROI on the microscope slide. As such, the visual inspection of a ROI on a microscope slide to identify potentially relevant pathology features is currently a time consuming and laborious process.

### SUMMARY OF THE INVENTION

This disclosure relates to a pathology inspection system and specifically to an automated method for detecting regions of interest in digital pathology images captured by a whole slide scanner and providing automated microscope control and/or augmented reality guidance to assist a pathologist in analyzing a microscope slide. An embodiment of the pathology inspection system includes a region identification (RI) model that extracts features of a whole slide image based on machine learning algorithms and generates feature vectors for each of the extracted features. The RI model then detects relevant regions of interests (ROIs) on a whole slide image of a microscope slide based on the feature vectors and establishes a unified coordinate system between the whole slide scanner and a microscope which can be used to visually inspect a ROI in greater detail. The RI model then generates coordinates in the coordinate system for each of the detected ROIs on the whole slide image and feature detection scores indicating the probability of the presence of relevant features on the whole slide image. Once the microscope slide has been unloaded from the whole slide scanner and loaded into the microscope, the pathology inspection system instructs a microscope stage controller of the microscope to translate the microscope stage, such that a detected ROI is centered in the field of view (FOV) of the microscope. The microscope then captures a FOV image of the ROI. FOV images are captured according to this method for each of the detected ROIs on the microscope slide, according to an embodiment.

The FOV images and the feature detection scores are provided to a grading model of the pathology inspection system which analyzes the FOV images, generating a set of pathology scores for each detected ROI. At least one of the pathology scores may indicate a probability of a presence of a disease in a patient or other pathology metric associated with the microscope slide, according to an embodiment. Information relating to the pathology score, the locations of the ROIs, the detected features, or other information may be displayed together with the FOV or whole slide image (e.g., as overlaid information) to provide guidance to a pathologist in analyzing the slide. The pathology inspection system improves the ease and speed with which a pathologist and/or user of the pathology inspection system inspects microscope slides in order to assess the medical condition of the patient.

Systems and methods for digital pathology in accordance with embodiments of the invention are illustrated. One embodiment includes a method that obtains a whole slide image of a microscope slide that includes a registration mark with a whole slide imaging device, wherein the registration mark is associated with an origin of a coordinate system. The method inputs the whole slide image into a region identification (RI) model to extract features of the whole slide image and generate feature vectors for the extracted features, detects a presence of a region of interest (ROI) based on the feature vectors, determines a set of coordinates of the ROI in the coordinate system, and translates a microscope stage of a microscope holding the microscope slide to a position corresponding to the coordinates of the ROI. The method captures a field of view (FOV) image with the microscope, wherein the FOV image includes at least a portion of the ROI. The method inputs the FOV image into a grading model to determine a pathology score that indicates a likelihood of a presence of a disease, and displays the FOV image and the pathology score on a display device.

In a further embodiment, the method further includes steps for marking the microscope slide with the registration mark, wherein the registration mark includes an etched pattern in the microscope slide.

In still another embodiment, the method further includes steps for displaying the whole slide image on the display device.

In a still further embodiment, the registration mark is configured so as to define the coordinate system as having sub-micron resolution.

In yet another embodiment, the translating the microscope stage includes changing a level of magnification of the microscope.

In a yet further embodiment, the displaying the pathology score on the display device further includes concurrently displaying the pathology score with the whole slide image, wherein the pathology score is displayed in a region corresponding to the ROI and overlapping a region where the whole slide image is displayed.

In another additional embodiment, the displaying the pathology score further includes concurrently displaying with the FOV image, wherein the pathology score is displayed in a region overlapping a region where the FOV image is displayed.

In a further additional embodiment, the method further includes steps for generating an annotation associated with the whole slide image and the FOV image, the annotation includes any combination of the feature vectors, the pathology score, the coordinates of the ROI, and a text string description inputted by a user, and storing the whole slide image, the FOV image, and the associated annotation.

In another embodiment again, the method further includes steps for pre-processing the whole slide image using at least one technique from a group consisting of image denoising, color enhancement, uniform aspect ratio, rescaling, normalization, segmentation, cropping, object detection, dimensionality deduction/increment, brightness adjustment, and data augmentation techniques, image shifting, flipping, zoom in/out, and rotation.

In a further embodiment again, the RI model comprises a set of RI model coefficients trained using a first set of whole slide training images, a second set of whole slide training images, a set of training ROI coordinates, and a function relating one of the whole slide images and the RI model coefficients to the presence of the ROI and the coordinates of the ROI, wherein each of the first set of whole slide training images includes a registration mark and at least one ROI, and each of the training ROI coordinates corresponds to the at least one ROI of one of the whole slide training images.

In still yet another embodiment, the first set of whole slide training images and the second set of whole slide training images are captured with the whole slide imaging device.

In a still yet further embodiment, the grading model comprises a set of grading model coefficients trained using a set of FOV training images, each of the FOV training images includes an RI identified by inputting a whole slide training image into the RI model a set of training pathology scores each corresponding to one of the whole slide training images, and a function relating one of the FOV training images and the grading model coefficients to the pathology score.

In still another additional embodiment, the set of FOV training images are captured with the microscope.

In a still further additional embodiment, the extracted features includes at least one of nuclei, lymphocytes, immune checkpoints, and mitosis events.

In still another embodiment again, the extracted features includes at least one of scale-invariant feature transform (SIFT) features, speeded-up robust features (SURF), and oriented FAST and BRIEF (ORB) features.

In a still further embodiment again, the RI model is a convolutional neural network.

In yet another additional embodiment, the RI model uses a combination of phase stretch transform, Canny methods, and Gabor filter banks to extract the features of the whole slide image and generate the feature vectors.

In a yet further additional embodiment, the ROI is a region encompassing a single cell.

In yet another embodiment again, the ROI is a region smaller than a single cell.

One embodiment includes a non-transitory machine readable medium containing processor instructions, where execution of the instructions by a processor causes the processor to perform a process that obtains a whole slide image of a microscope slide includes a registration mark with a whole slide imaging device, wherein the registration mark is associated with an origin of a coordinate system. The process inputs the whole slide image into a region identification (RI) model to extract features of the whole slide image and generate feature vectors for the extracted features, detects a presence of a region of interest (ROI) based on the feature vectors, determines a set of coordinates of the ROI in the coordinate system, and translates a microscope stage of a microscope holding the microscope slide to a position corresponding to the coordinates of the ROI. The process captures a field of view (FOV) image with the microscope, wherein the FOV image includes at least a portion of the ROI. The process inputs the FOV image into a grading model to determine a pathology score that indicates a likelihood of a presence of a disease, and displays the FOV image and the pathology score on a display device.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 shows a region of interest (ROI) inspection system for identifying regions of interest in digital images of a microscope slide and providing automated guidance to assist a pathologist in visual inspection of the regions of interest, according to one embodiment

FIG. 2 is an interaction diagram illustrating interactions between components of the ROI inspection system, according to one embodiment.

FIG. 3 illustrates a process for training a region identification (RI) model, according to one embodiment.

FIG. 4 illustrates a process for generating sets of ROI coordinates identifying ROIs and sets of feature detection scores corresponding to the ROIs using an RI model, according to one embodiment.

FIG. 5 illustrates a process for training a grading model, according to one embodiment.

FIG. 6 illustrates a process for generating a pathology score for a field of view (FOV) image that includes a ROI using a grading model, according to one embodiment.

FIG. 7 illustrates an example of using feature detection via a phase stretch transform (PST) to generate a set of ROI coordinates using a region identification model, according to one embodiment.

FIG. 8 is a flowchart illustrating a process for detecting ROIs on a microscope slide using the ROI inspection system and providing automated guidance associated with the ROIs, according to one embodiment.

FIG. 9 is a high-level block diagram illustrating an example of a computing device used either as client device, application server, and/or database server, according to one embodiment.

### DETAILED DESCRIPTION

### System Architecture

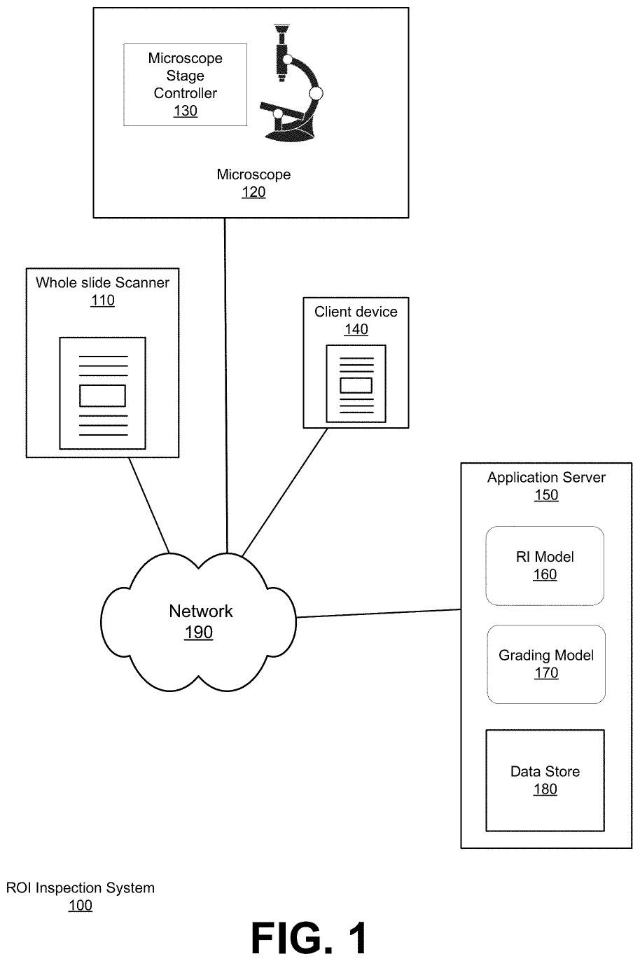

FIG. 1 shows a region of interest (ROI) inspection system 100 for identifying ROIs on a microscope slide corresponding to regions that are likely to be of interest to a pathologist, and for providing automated control of a microscope and/or augmented reality guidance to assist a pathologist in analyzing the identified ROIs.

The ROI inspection system 100 includes a whole slide scanner 110, a client computing device 140, and an application server 150 coupled by a network 190. ROI inspection system 100 further includes a microscope 120 with a computer-controlled microscope stage controller 130. The application server 150 comprises a region identification (RI) model 160, a grading model 170, and a data store 180, according to some embodiments. Although FIG. 1 illustrates only a single instance of most of the components of the ROI inspection system 100, in practice more than one of each component may be present, and additional or fewer components may be used. In a variety of embodiments, RI models and/or grading models can be implemented as part of a client device, where functions of the RI models and/or grading models can be performed locally on the client device.

According to some embodiments, the microscope slide for analyzing by a ROI inspection system can be a microscope slide that includes a sample of tissue, cell, or body fluids for visual inspection. In several embodiments, microscope slides can include immunohistochemistry (IHC) slides that include one or more stains to improve visualization of particular proteins or other biomarkers. Microscope slides in accordance with certain embodiments of the invention can include a fluorescent multiplexed imaging (e.g., mIHC) slide.

A ROI may be a region of the microscope slide that includes visual features relevant to a diagnosis of a patient associated with the microscope slide, and thus may represent a ROI to a pathologist or other medical professional. For example, ROIs may be used in assessing a Gleason score indicating a severity of prostate cancer for a patient associated with the microscope slide. In certain embodiments, ROI inspection systems can analyze whole slide images captured by a whole slide scanner to detect one or more ROIs within the whole slide image, corresponding to regions on the microscope slide relevant to diagnosing a patient associated with the microscope slide.

ROIs identified by the ROI inspection system may have varying size depending on the type of slide and the particular features that may be of interest. For example, in an embodiment, the ROIs may be regions containing at least 10 cells. In other embodiments, the ROI may be a region containing at least 2, 100, 1000, 10,000, 100,000, or 1 million cells. In another embodiment, ROIs can be regions containing a single cell. In another embodiment, the ROI is a region encompassing an organelle within a single cell.

ROIs in accordance with several embodiments of the invention can be identified by a single set of coordinates that indicate a point (e.g., the center) of a given ROI. In many embodiments, ROIs may be identified by a set of ROI coordinates that represent a bounding shape of a corresponding ROI. In many embodiments, the boundary of a ROI can be a rectangle. In other embodiments, the boundary of the ROI may an arbitrary shape. Although ROIs are described as regions (or coordinates) of a microscope slide, one skilled in the art will recognize that ROIs may often be identified in images of the slide, without departing from this invention.

### Whole Slide Scanner

Whole slide scanners in accordance with many embodiments of the invention are devices configured to generate a digital whole slide image of an entire microscope slide. In numerous embodiments, whole slide scanners may capture the whole slide image in high resolution. According to some embodiments, whole slide scanners can capture whole slide images in stereoscopic 3D. In other embodiments, whole slide scanners can capture fluorescent whole slide images by stimulating fluorescent specimens on the microscope slide with a low wavelength light source and detecting emitted light from the fluorescent specimens. Whole slide scanners in accordance with a number of embodiments of the invention may include a white light source, a narrowband light source, a structured light source, other types of light sources, or some combination thereof, to illuminate the microscope slide for imaging. In certain embodiments, whole slide scanners may, for example, include an image sensor such as a charge coupled device (CCD) sensor, a complementary metal-oxide semiconductor (CMOS) sensor, other types of image sensors, or some combination thereof, configured to image light reflected and/or emitted from the microscope slide. Whole slide scanners in accordance with many embodiments of the invention may also include optical elements, for example, such as lenses, configured to condition and/or direct light from the microscope slide to the image sensor. Optical elements of whole slide scanners in accordance with certain embodiments of the invention may include mirrors, beam splitters, filters, other optical elements, or some combination thereof.

In certain embodiments, microscope slides may be marked with registration marks that are detectable in whole slide images of a microscope slide. This can enable establishment of a coordinate system such that particular pixels of the whole slide image can be mapped to physical locations on the microscope slide based on respective distances from the registration marks, as will be described in further detail below.

### Microscope

Microscopes are devices that can include a motorized microscope stage, a microscope stage controller, one or more optical elements, one or more light sources, and one or more image sensors, among other components, and image portions of a microscope slide in the form of field of view (FOV) images. Microscope stages can comprise a movable mounting component that holds a microscope slide in a position for viewing by the optical components of the microscope. Microscope stage controllers may control the position of a microscope stage based on manual inputs or may accept instructions from a connected computing device (e.g., a client device or application server) that cause the microscope stage to translate its position in three dimensions. In a variety of embodiments, microscope stage controllers may also accept instructions to adjust the magnification, focus, other properties of the microscope, or some combination thereof. For example, microscope stage controllers may receive coordinates identifying a particular ROI and may configure the physical position, magnification, focus, or other properties of the microscope to enable the microscope to obtain a FOV image corresponding to that ROI.

In an embodiment, microscope stage controllers may control the physical position, magnification, focus or other properties of the microscope based on a coordinate system established for the whole slide image. For example, particular pixels of the whole slide image may be mapped to physical locations on the microscope slide based on distances from registration marks on the microscope slides so that the microscope controller can control the microscope to produce a FOV image corresponding to particular coordinates in a common coordinate system.

In numerous embodiments, microscopes can capture FOV images at magnifications equal to and/or greater than a whole slide scanner. In various embodiments, the microscope may be, specifically, a pathology microscope. In varying embodiments, the microscope is one of: a bright field microscope, a phase contrast microscope, a differential interference contrast microscope, a super resolution microscope, and a single molecule microscope.

### Client Device

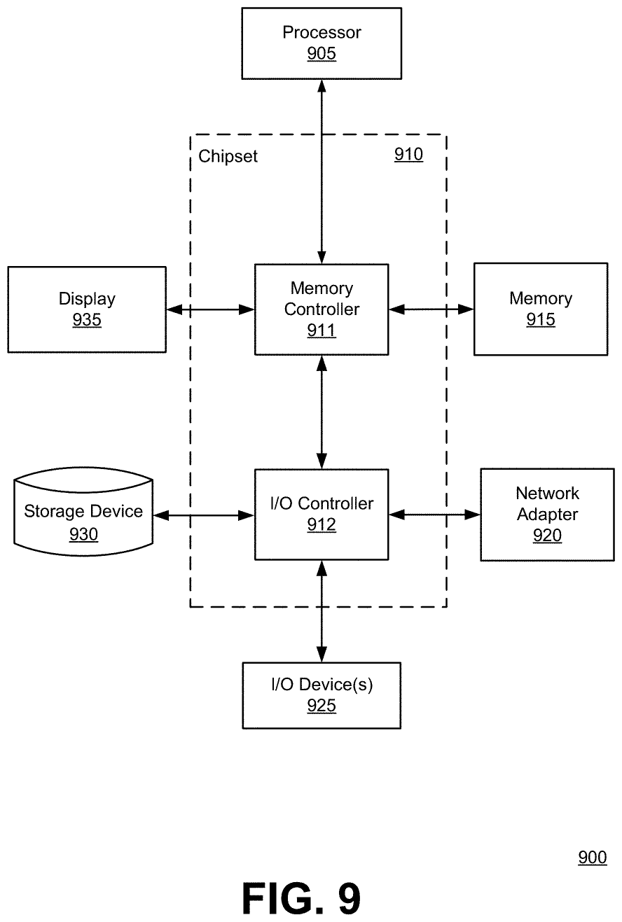

Client devices in accordance with several embodiments of the invention comprise a computer system that may include a display and input controls that enable a user to interact with a user interface for analyzing the microscope slides. An exemplary physical implementation is described more completely below with respect to FIG. 9. In a number of embodiments, client devices can be configured to communicate with other components of a ROI inspection system via a network to exchange information relevant to analyzing microscope slides. For example, client devices in accordance with numerous embodiments of the invention may receive a digitized whole slide image captured by a whole slide scanner for display on the display of the client device. Furthermore, client devices in accordance with some embodiments of the invention may receive digital FOV images obtained by a microscope for display. In a number of embodiments, client devices may display additional information relating to analysis of a microscope slide such as, for example, overlaying information on a FOV image relating to observed features, a diagnosis prediction, and/or other relevant information. Client devices in accordance with a variety of embodiments of the invention may enable a user to provide various control inputs relevant to operation of components of the ROI inspection system.

In some embodiments, client devices may also perform some data and image processing on the whole slide image and/or FOV images obtained by a client device locally using the resources of client device. Client devices in accordance with some embodiments of the invention may communicate processed images to an application server via a network.

Client devices in accordance with a number of embodiments of the invention may communicate with a whole slide scanner, microscope, and/or the application server using a network adapter and either a wired or wireless communication protocol, an example of which is the Bluetooth Low Energy (BTLE) protocol. BTLE is a short-range, low-powered, protocol standard that transmits data wirelessly over radio links in short range wireless networks. In other embodiments, other types of wireless connections are used (e.g., infrared, cellular, 4G, 5G, 802.11).

### Application Server

Application servers can be a computer or network of computers. Although a simplified example is illustrated in FIG. 9, typically application servers in accordance with several embodiments of the invention can be server class systems that use powerful processors, large memory, and faster network components compared to a typical computing system used, for example, as client devices. The server can include large secondary storage, for example, using a RAID (redundant array of independent disks) array and/or by establishing a relationship with an independent content delivery network (CDN) contracted to store, exchange and transmit data. Additionally, computing systems can include an operating system, for example, a UNIX operating system, LINUX operating system, or a WINDOWS operating system. Operating systems can manage the hardware and software resources of application servers and also provide various services, for example, process management, input/output of data, management of peripheral devices, and so on. The operating system can provide various functions for managing files stored on a device, for example, creating a new file, moving or copying files, transferring files to a remote system, and so on.

In numerous embodiments, application servers can include a software architecture for supporting access to and use of a ROI inspection system by many different client devices through a network, and thus in some embodiments can be generally characterized as a cloud-based system. Application servers in accordance with some embodiments of the invention can provide a platform (e.g., via client devices) for medical professionals to report images and data recorded by a whole slide scanner and a microscope associated with a microscope slide, a corresponding patient, and the patient's medical condition, collaborate on treatment plans, browse and obtain information relating to the patient's medical condition, and/or make use of a variety of other functions. Data of ROI inspection systems in accordance with several embodiments of the invention may be encrypted for security, password protected, and/or otherwise secured to meet all Health Insurance Portability and Accountability Act (HIPAA) requirements. In many embodiments, any analyses that incorporate data from multiple patients and are provided to users may be de-identified so that personally identifying information is removed to protect subject privacy.

Application servers in accordance with a variety of embodiments of the invention can provide a platform including RI models and/or grading models. For example, application servers may communicate with client devices to receive data including whole slide images and/or FOV images from a whole slide scanner, a microscope, the client device, or some combination thereof to provide as inputs to a RI model and/or grading model. In many embodiments, application servers can execute RI models and/or grading models to generate output data from the RI and/or grading models. In a variety of embodiments, application servers may then communicate with client devices to display the output data to a user of the ROI inspection system.

Application server can be designed to handle a wide variety of data. In certain embodiments, application servers can include logical routines that perform a variety of functions including (but not limited to) checking the validity of the incoming data, parsing and formatting the data if necessary, passing the processed data to a data store for storage, and/or confirming that a data store has been updated.

In many embodiments, application servers can receive whole slide images and FOV images from client devices and may apply a variety of routines on the received images as described below. Particularly, in the exemplary implementations described below, RI models and grading models execute routines to access whole slide images and FOV images, analyze the images and data, and output the results of its analysis to a client device for viewing by a medical professional.

### Region Identification Model

Region identification (RI) models in accordance with many embodiments of the invention can automatically detect relevant ROIs based on a whole slide image of a microscope slide. In a variety of embodiments, detected ROIs can be identified based on ROI coordinates in a coordinate system established based on registration marks on the slide. Registration marks in accordance with a variety of embodiments of the invention can be identified based on the physical slide and the whole slide image, such that particular pixels of the whole slide image (or locations of ROIs) can be mapped to physical locations on the microscope slide. In order to identify the ROIs, RI models in accordance with many embodiments of the invention can perform feature extraction on an input whole slide image to generate a feature vector and can detect the presence of a ROI based on the extracted features using one or more machine learning algorithms. Feature vectors in accordance with numerous embodiments of the invention can include (but are not limited to) outputs of one or more layers of a convolutional neural network that has been trained to classify images based on their labeled pathology results. For example, RI models in accordance with some embodiments of the invention can scan the whole slide image and classify different regions as ROIs if they include features meeting certain characteristics (e.g., characteristics learned to be representative of a particular pathology result).

The features extracted from the whole slide image may include anatomical features of a biological specimen that can be identified in the whole slide image based on visual analysis techniques. For example, features may include the presence and/or location of one or more tumor cells, the presence and/or location of one or more cell nuclei, the presence and/or location of one or more organelles of a cell, the orientation of a T cell relative to a tumor cell, T presence and/or location of one or more lymphocytes, the presence and/or location of one or more immune checkpoints, and the presence and/or location of one or more mitosis events, or some combination thereof. Features may also include lower level features such as edges and textures in the images. These types of features may be extracted using, for example, a phase stretch transform (PST) based algorithm, a Canny transform, a Sobel transform, or other transform. According to one embodiment, the extracted features may include at least one of: scale-invariant feature transform (SIFT) features, speeded-up robust features (SURF), and oriented FAST and BRIEF (ORB) features.

In some embodiments, specialized pattern recognition processes (e.g., deep learning models such as a convolutional neural network (CNN)) may be used in RI models to identify ROIs with biological anomalies. Specialized pattern recognition processes (such as, but not limited to CNNs and/or graph and tree processes) may be used in accordance with a number of embodiments of the invention for various purposes such (but not limited to) analyzing morphology of tumor cells and immune cells, examining the relation between tumor cells and immune cells, identifying anomalies between responders and non-responders of tumor cells in relation to immune cells, performing ROI detection, and/or performing patient stratification to tailor targeted immune therapies, among other applications.

### Grading Model

In a variety of embodiments, grading models can generate a pathology score based on a set of one or more FOV images associated with a ROI of a microscope slide. Grading models in accordance with several embodiments of the invention may apply a machine-learned model to generate the score based on features of the FOV image. In various embodiments, the features utilized by grading models may incorporate the same features utilized by RI models, similar to those discussed above. Grading models in accordance with a number of embodiments of the invention may receive, as inputs, one or more feature vectors generated from a RI model for re-use by the grading model. The generated pathology scores may indicate, for example, a predicted severity of a disease. In a variety of embodiments, the determined pathology scores can be provided to a client device for display as, for example, an overlay on an FOV image being viewed on the display. In certain embodiments, the determined pathology scores can be used to determine appropriate treatments for a patient.

### Data Store

Data stores in accordance with some embodiments of the invention can store whole slide images used as inputs for RI models, FOV images corresponding to ROIs used as inputs for grading models, output data from RI models and/or grading model, and/or related patient and medical professional data. Patient and medical professional data may be encrypted for security and is at least password protected and otherwise secured to meet all applicable Health Insurance Portability and Accountability Act (HIPAA) requirements. In certain embodiments, data stores can remove personally identifying information from patient data used in any analyses that are provided to users to protect patient privacy.

Data stores in accordance with many embodiments of the invention may be a hardware component that is part of a server, such as an application server as seen in FIG. 1, such that the data store is implemented as one or more persistent storage devices, with the software application layer for interfacing with the stored data in the data store. Although the data store 180 is illustrated in FIG. 1 as being included in the application server 150, the data store 180 may also be a separate entity from the application server 150.

Data stores in accordance with various embodiments of the invention can store data according to defined database schemas. Typically, data storage schemas across different data sources vary significantly even when storing the same type of data including cloud application event logs and log metrics, due to implementation differences in the underlying database structure. Data stores may also store different types of data such as structured data, unstructured data, or semi-structured data. Data in the data store may be associated with users, groups of users, and/or entities. Data stores can provide support for database queries in a query language (e.g., SQL for relational databases, JSON for NoSQL databases, etc.) for specifying instructions to manage database objects represented by the data store, read information from the data store, and/or write to the data store.

### Network

Networks can include various wired and wireless communication pathways between devices of a ROI system, such as (but not limited to) client devices, whole slide scanners, microscopes, application servers, and data stores. Networks can use standard Internet communications technologies and/or protocols, such as (but not limited to) Ethernet, IEEE 802.11, integrated services digital network (ISDN), asynchronous transfer mode (ATM), etc. Similarly, the networking protocols can include (but are not limited to) the transmission control protocol/Internet protocol (TCP/IP), the hypertext transport protocol (HTTP), the simple mail transfer protocol (SMTP), the file transfer protocol (FTP), etc. The data exchanged over a network can be represented using technologies and/or formats including (but not limited to) the hypertext markup language (HTML), the extensible markup language (XML), etc. In addition, all or some links can be encrypted using various encryption technologies such as (but not limited to) the secure sockets layer (SSL), Secure HTTP (HTTPS) and/or virtual private networks (VPNs). In some embodiments, custom and/or dedicated data communications technologies can be implemented instead of, or in addition to, the ones described above.

In a number of embodiments, networks can comprise a secure network for handling sensitive or confidential information such as, for example, protected health information (PHI). For example, networks and/or the devices connected to them may be designed to provide for restricted data access, encryption of data, and otherwise may be compliant with medical information protection regulations such as HIPAA.

Although client devices, whole slide scanners, and microscopes are described above as being separate physical devices (such as a computing device, a whole slide scanner, and a microscope, respectively), one skilled in the art will recognize that the functions of the various devices can be distributed among more devices and/or consolidated into fewer devices without departing from this invention. For example, a whole slide scanner may include an audiovisual interface including a display or other lighting elements as well as speakers for presenting audible information. In such an implementation, the whole slide scanner itself may present the contents of information obtained from the application server, such as the ROI coordinates and grading score determined by the ROI inspection system, in place of or in addition to presenting them through the client devices. In another example, a microscope may include the audiovisual interface including a display or other lighting elements as well as speakers for presenting audible information. In such an implementation the microscope itself may present the contents of information obtained from the application server, such as the ROI coordinates and grading score determined by the ROI inspection system, in place of or in addition to presenting them through the client devices. In another example, a whole slide scanner, a microscope, and a client device are integrated into a single device including the audiovisual interface.

### Data Flow

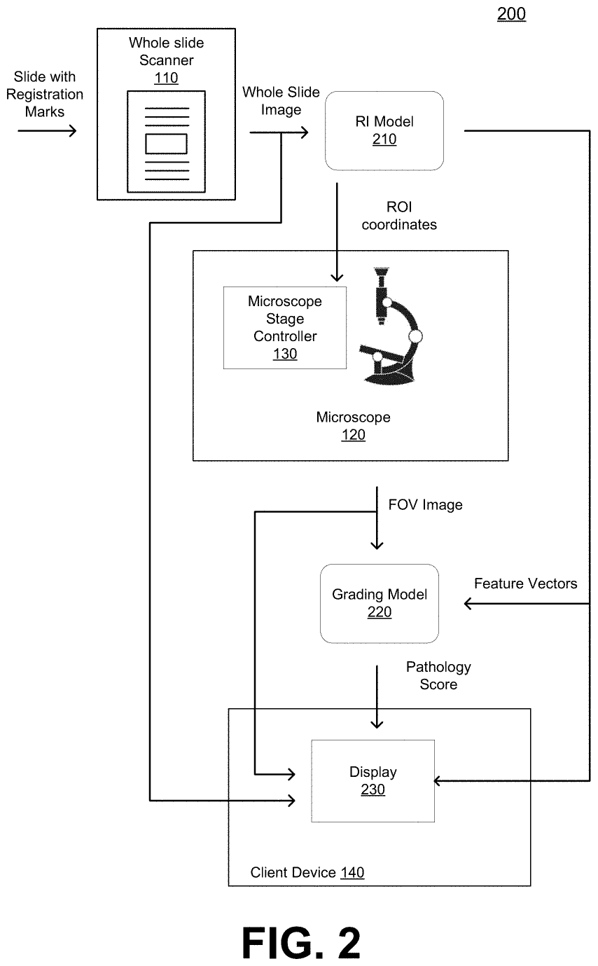

FIG. 2 is an interaction diagram representing a process for identifying ROIs of a microscope slide and subsequent image capture and analysis of the ROI in accordance with an embodiment of the invention. As illustrated in FIG. 2, a microscope slide containing a sample for visual analysis is loaded into the whole slide scanner 110. The microscope slide may include registration marks comprising visible indicators on a surface of the microscope. For example, the registration marks may be a set of shapes and/or a pattern that is etched into a surface of the microscope slide. In various embodiments, registration marks can be applied to a surface of the microscope slide via an opaque ink and/or dye. In many embodiments, ROI inspection systems can apply the registration marks to the microscope slide via laser etching a pattern into a surface of the microscope slide. In other embodiments, the microscope slides can be pre-marked with registration marks.

Registration marks can enable alignment of a coordinate system between the different components of an ROI inspection system. The coordinate system provides a common spatial reference for describing the location of a detected ROI, feature, and/or region on the microscope slide and in the whole slide image. In numerous embodiments, the coordinate system can be a cartesian coordinate system including 2 orthogonal axes, for example an x-axis and a y-axis. The coordinate system in accordance with a variety of embodiments of the invention can be a polar coordinate system including a distance or radius coordinate and an angular coordinate. In other embodiments, different types of coordinate systems may be used. In some embodiments, the coordinate system has sub-micron resolution. Coordinate systems in accordance with a variety of embodiments of the invention can include 3D coordinates that includes a depth coordinate (e.g., for directing the focus of a microscope) along a z-axis.

Whole slide scanners in accordance with a number of embodiments of the invention can capture a whole slide image that is provided to a trained region identification (RI) model and to a display of a client device. In many embodiments, whole slide images can be segmented by a RI model and feature extraction is performed on each of the segments. RI models in accordance with some embodiments of the invention can generate a feature vector for each of the regions of the whole slide image representing the identified features. In many embodiments, RI models can apply a function to the one or more feature vectors to detect a set of ROIs of the microscope slide. RI models in accordance with a variety of embodiments of the invention may apply a classification function to feature vectors associated with each segment of the whole slide image to classify whether or not the segment meets criteria for being classified for inclusion in a ROI. Alternatively, or conjunctively, machine learning models may be applied on the entire whole slide image to directly identify the ROIs from the feature vectors without necessarily segmenting the image. In several embodiments, RI models can generate corresponding sets of ROI coordinates in the coordinate system for each detected ROI to indicate the respective locations. In various embodiments, ROI coordinates can be generated based on identified features from the whole slide image and/or segments of the whole slide image. ROI coordinates in accordance with a variety of embodiments of the invention can identify a location (e.g., bounding boxes, a center point, etc.) in an image and map the location to coordinates for a ROI on a slide.

In a number of embodiments, ROI coordinates can be generated using a trained model that learns using a set of training images (using manual annotations). Trained models in accordance with a number of embodiments of the invention can be deep neural networks or computationally inexpensive alternatives such as anchored point regression and light learned filter techniques. Methods for anchored point regression and light learned filter techniques in accordance with various embodiments of the invention are described in “RAISR: Rapid and accurate image super resolution” by Romano et al., published Nov. 15, 2016, and “Fast Super-Resolution in MRI Images Using Phase Stretch Transform, Anchored Point Regression and Zero-Data Learning” by He et al., published Sep. 22, 2019, the disclosures of which are incorporated by reference herein in their entirety.

In many embodiments, feature extraction techniques used by RI models may include (but are not limited to) one or more of: techniques based on edge detection, PST, an algorithm based on PST, the Canny methods, Gabor filter banks, or any combination thereof. In numerous embodiments, resolution enhancement can be performed on the whole slide image prior to feature extraction using PST, any of the above algorithms, other suitable algorithms, or some combination thereof. Additional details of a phase stretch transform may be found in, e.g., U.S. patent application Ser. No. 15/341,789 which is hereby incorporated by reference in its entirety.

In many embodiments, ROI coordinates can be provided as inputs for providing instructions to the microscope stage controller to locate a ROI in the microscope's FOV. Grading models in accordance with many embodiments of the invention can receive ROI coordinates in order to generate pathology scores for each ROI. In a variety of embodiments, feature detection scores may also be provided to grading models and client devices as inputs for generating pathology scores for a detected ROI on the microscope slide and for displaying the feature detection scores to a pathologist and/or user of a ROI inspection system.

The microscope slide can be subsequently unloaded from the whole slide scanner and loaded into the microscope. In many embodiments, the unloading and loading of the microscope slide can be performed manually by a user of the ROI inspection system. Client devices may notify the user when to unload the microscope slide from the whole slide scanner and load the microscope slide into the microscope after the whole slide scanner has finished scanning the microscope slide. In numerous embodiments, the loading and unloading of the microscope slide can be carried out automatically after the detection of all ROI regions on the microscope slide by the RI model. The automatic unloading and loading of the microscope slide may be carried out, for example, by a series of mechanical stages that exchange the microscope slide between the whole slide scanner and the microscope.

According to some embodiments, the microscope stage may initially be set to a preset initial position upon loading the microscope slide. In various embodiments, to calibrate the microscope position, the microscope can capture one or more FOV images of the microscope slide at the initial position, with the registration marks within the FOV. The images may be analyzed by the client device to determine if the microscope stage is correctly set to the preset initial position based on the location of the registration marks within the one or more FOV images. In a variety of embodiments, the microscope stage may be set to a rough position with the registration marks within the FOV, and the client device may subsequently instruct the microscope stage controller to perform fine adjustments, translating the microscope stage to the preset initial position such that the registration marks appear at a specific location within the FOV of the microscope, within a threshold degree of error. After the microscope stage has been set to the preset initial position, the client device in accordance with various embodiments of the invention can instruct the microscope stage controller to translate the microscope stage to a position such that the detected ROI is centered in the FOV of the microscope, based on the ROI coordinates and the relative position of the registration marks. In some embodiments, the microscope stage controller can also change the magnification and/or the focus of the microscope based on the coordinates of the ROI.

Once an ROI is positioned (e.g., centered) in the FOV of the microscope, the microscope can capture one or more FOV images of the ROI. In a number of embodiments, the microscope can capture FOV images at a greater magnification than was used when capturing the whole slide images with the whole slide scanner. In several embodiments, a pathologist using the ROI inspection system may manually inspect the ROI using the microscope after the FOV images are captured. According to another embodiment, the pathologist may manually perform fine adjustments, translating the microscope stage and adjusting the magnification and focus of the microscope, after the microscope stage controller has positioned the ROI in the FOV of the microscope. In several embodiments, the microscope can provide the one or more FOV images to a trained grading model and to the client device for display.

Grading models in accordance with certain embodiments of the invention can receive as inputs one or more FOV images of an ROI from the microscope, as well as a set of feature vectors associated with the ROI from the RI model 210. Feature vectors associated with the ROI can include (but are not limited to) feature vectors from passing a FOV image through a machine learning model, feature vectors used to identify the ROIs, etc. In many embodiments, grading models can generate one or more pathology scores. Pathology scores in accordance with some embodiments of the invention may be computed based on a machine learned model applied to the FOV image, the feature vectors, or a combination thereof. In certain embodiments, pathology scores may correspond to a probability of a presence of a pathology feature in the ROI, a severity of a pathology feature, or other metric associated with a pathology feature. In one embodiment, at least one of the pathology scores corresponds to a Gleason Score representing a predicted aggressiveness of a prostate cancer. In other embodiments, at least one of the pathology scores may correspond to a predicted presence or absence of a disease.

In certain embodiments, grading models can provide the pathology score to client devices. The client device 140 includes a display 230 on the client device 140, on which a pathologist or other user of the ROI inspection system 100 may view the whole slide images, the FOV images, and/or the pathology scores. In some embodiments, whole slide images can be shown on a display with the locations of the detected ROIs indicated by an augmented reality interface overlaid on the whole slide image. In some embodiments, pathology scores for each detected ROI can be overlaid on the whole slide image, indicating the pathology score that was determined for each detected ROI. In some embodiments, FOV images of each ROI may be shown on the display with the location of the detected ROIs and a determined pathology score for the ROI in the FOV image indicated by an augmented reality interface overlaid on the FOV image. Detected visual features in accordance with various embodiments of the invention can be indicated by the augmented reality interface overlaid on the FOV image. In many embodiments, microscopes can continuously provide FOV images to a client device as a pathologist manually translates the microscope stage and adjusts the magnification and focus of the microscope to examine a detected ROI in detail. The display in accordance with a variety of embodiments of the invention may continue to overlay the locations of the detected ROIs and the determined pathology score for a ROI in the FOV of the microscope via an augmented reality interface.

In one embodiment, the whole slide images and FOV images are visible light images. However, in other embodiments, the whole slide images and FOV images may be images captured in various wavelengths, including but not limited to the visible light spectrum. For example, the whole slide images and FOV images may be captured in a range of infrared wavelengths.

Images captured by whole slide scanners and/or microscopes may be curated, centered, and cropped by an image pre-processing algorithm that assesses the quality and suitability of these images for use in a RI model and/or a grading model. The image pre-processing may be performed by client devices before the images are provided to a RI model and/or a display. In many embodiments, the image pre-processing can performed by application servers. Good image pre-processing can lead to a robust AI model for accurate predictions. Pre-processing techniques that may be performed on the images may include (but are not limited to): image denoising, color enhancement, uniform aspect ratio, rescaling, normalization, segmentation, cropping, object detection, dimensionality deduction/increment, brightness adjustment, and/or data augmentation techniques to increase the data size like (but not limited to): image shifting, flipping, zoom in/out, rotation etc., determining quality of the image to exclude bad images from being a part of training dataset, and/or image pixel correction.

### RI Model

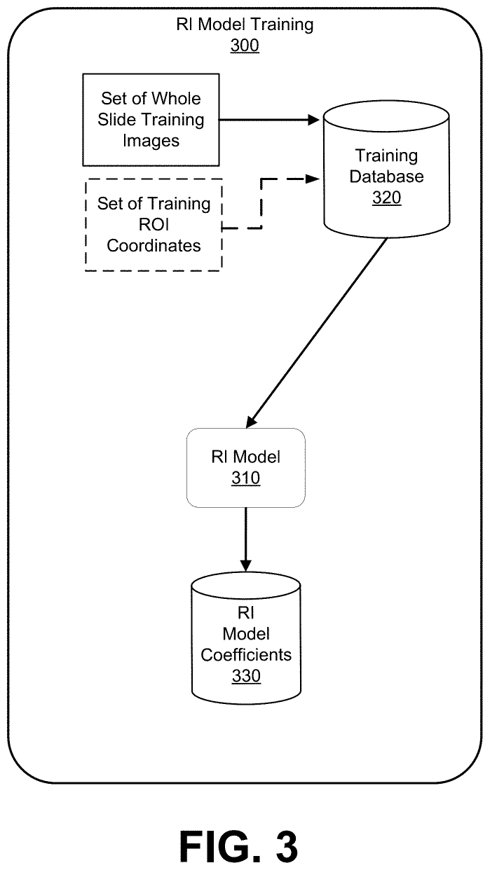

FIG. 3 illustrates a process for training 300 the region identification (RI) model 310, according to one embodiment. In numerous embodiments, RI models can also perform resolution enhancement of the whole slide image. The RI model 310 is trained on a set of whole slide training images of microscope slides that may depict various pathology results. In certain embodiments, at least a subset of the microscope slides includes one or more ROIs associated with a pathology result of interest. In many embodiments, in the case of supervised learning, ROI coordinates identifying locations of the ROIs in the whole slide images may also be included in the training set as labels. Here, the coordinates may be obtained based on a visual inspection performed by a pathologist according to traditional evaluation of microscope slides.

In various embodiments, training data including the whole slide training images and the training ROI coordinates if present are stored in a training database 320 that provides the training data to the RI model 310. The RI model 310 may further update the training database 320 with new whole slide images (and ROI coordinate labels) on a rolling basis as new input whole slide images are obtained, the images are analyzed, and ROIs are identified.

The training of RI models in accordance with certain embodiments of the invention may be performed using supervised machine learning techniques, unsupervised machine learning techniques, or some combination thereof. For example, training of a RI model to perform tasks such as nuclei segmentation may be supervised, while simultaneously training of the RI model to perform tasks such as pattern recognition for cancer detection and profiling may be unsupervised. Examples of unsupervised training algorithms include K means clustering and principal component analysis.

In the case of unsupervised learning, the training coordinates may be omitted as inputs and the locations of the ROIs are not expressly labeled. In this example, the RI model can pre-process the input whole slide training images as described above and can perform feature extraction on the pre-processed images. The RI model in accordance with various embodiments of the invention can learn similarities between extracted feature vectors and clusters of regions having similar feature vectors (e.g., regions corresponding to ROIs and regions without any pathology features of interest).

In this example, the RI model 310 learns RI model coefficients 330 that, when applied by a classification function of the RI model 310, can classify an input image into one of the learned clusters.

In the case of supervised learning, RI models in accordance with a number of embodiments of the invention can learn model coefficients (or weights) that best represent the relationship between each of the whole slide training images input into a function of the RI model and ROIs on the microscope slide associated with the whole slide training images. In certain embodiments, RI models can learn RI model coefficients according to specialized pattern recognition algorithms such as (but not limited to) a convolutional neural network (CNN). In other embodiments, RI models can apply graph- and tree-based algorithms.

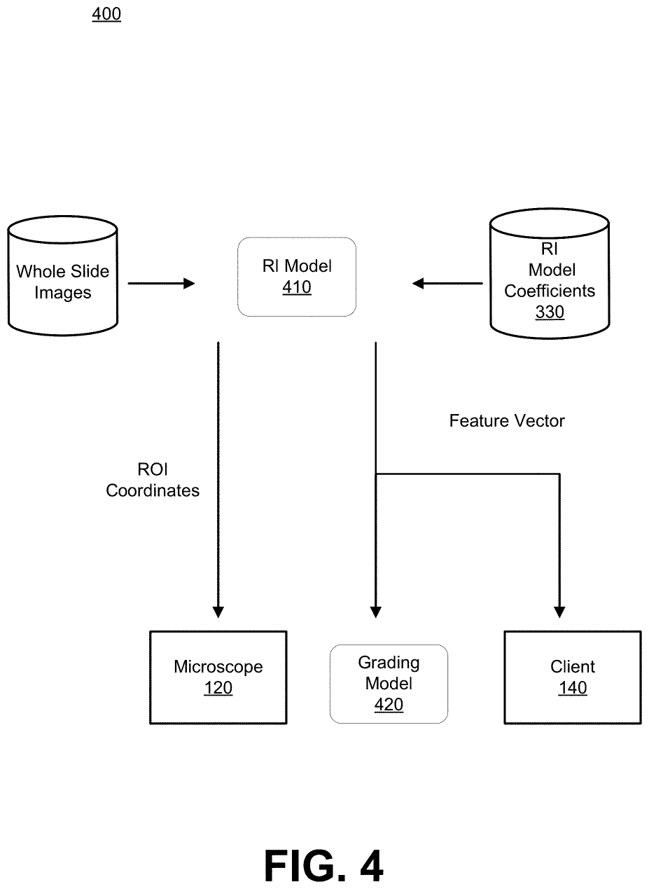

Once the RI model coefficients are known, RI models in accordance with numerous embodiments of the invention can be used, as discussed in FIGS. 2 and 4, by accessing the trained RI model coefficients and a function specified by the model, and inputting input values of the RI model coefficients into the function to detect a set of ROIs. For each detected ROI, the RI model can output a set of ROI coordinates for the ROI. In various embodiments, ROI coordinates can be generated based on identified features from the whole slide image and/or segments of the whole slide image. ROI coordinates in accordance with a variety of embodiments of the invention can identify a location (e.g., bounding boxes, a center point, etc.) in an image and map the location to coordinates for a ROI on a slide.

FIG. 4 illustrates an example of a process 400 for generating sets of ROI coordinates and sets of feature vectors corresponding to ROIs on a whole slide image using a RI model 410, according to one embodiment. The trained ROI model 410 receives as input one or more whole slide images of a microscope slide captured with the whole slide scanner 110. The RI model 410 accesses the trained RI model coefficients 330, extracts features of the input whole slide images to generates feature vectors, and applies the learned RI model 410 to detect ROIs. The RI model 410 outputs the set of ROI coordinates for each of the detected ROIs. In various embodiments, generated feature vectors may furthermore be outputted for re-use by the grading model 420 and/or may be outputted to the client device to be integrated into a display of an FOV image corresponding to a detected ROI.

### Grading Model

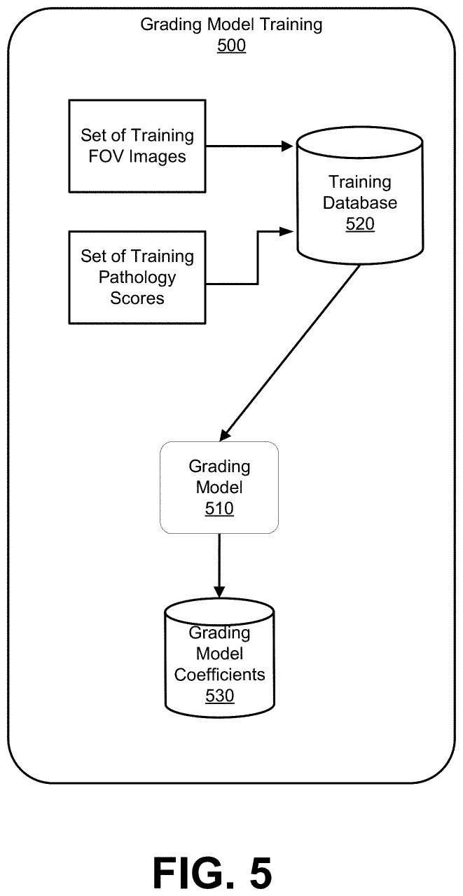

FIG. 5 illustrates a process for training 500 of a grading model, according to one embodiment. Grading models in accordance with a number of embodiments of the invention can be trained on a set of training FOV images. In the case of supervised learning, a corresponding set of training pathology scores associated with each of the training FOV images may also be inputted as labels. In some embodiments, the training data including the training FOV images and the pathology scores, if present, can be stored in a training database. Training databases in accordance with certain embodiments of the invention may be updated with new FOV images (and pathology score labels) on a rolling basis as new FOV images are obtained, the images are analyzed, and scores are assigned.

Training in accordance with numerous embodiments of the invention can generate training feature vectors from the set of training FOV images and/or the set of training pathology scores. In a variety of embodiments, the training feature vectors used in training of a grading model may include the some or all of the same training feature vectors obtained in training of a RI model.

In the case of supervised learning, the training pathology scores may be determined by a pathologist visually inspecting each training FOV image. The pathologist may use traditional diagnostic techniques in determining the training pathology scores.

Grading models in accordance with some embodiments of the invention can learn grading model coefficients (or weights) based on the training images and the training pathology scores. In some embodiments, grading model coefficients can be determined so as to best represent the relationship between each of the training FOV images input into a function of the grading model and their corresponding training pathology scores.

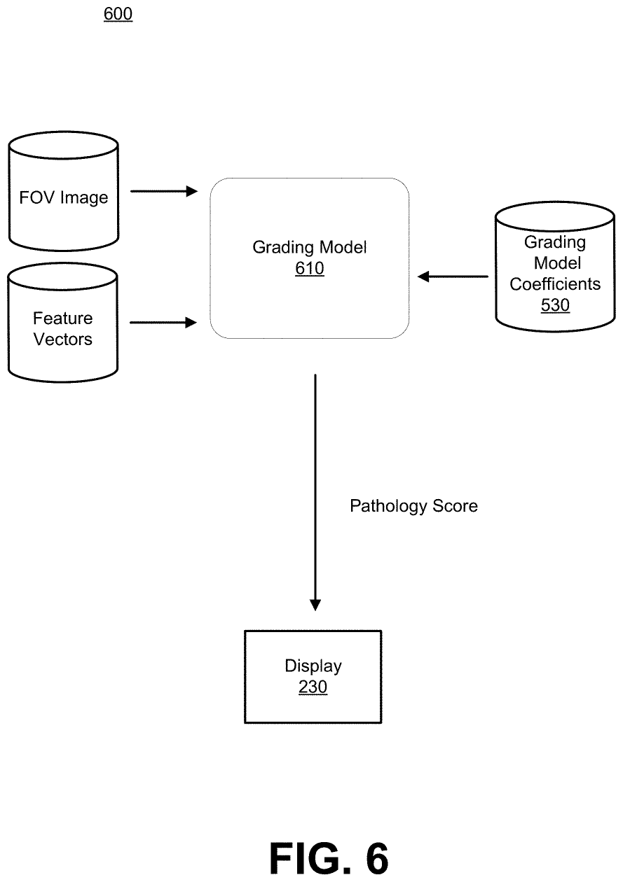

Once the grading model coefficients are known, grading models in accordance with certain embodiments of the invention may be used to generate pathology scores for FOV images. In a number of embodiments, grading models may be used for prediction (e.g., as described with reference to FIG. 6) by accessing the trained grading model coefficients and the function specified by the model, and inputting input values for the grading model coefficients to generate a set of pathology scores for a ROI depicted in an input FOV image.

FIG. 6 illustrates a process 600 for generating a pathology score, according to one embodiment. The trained grading model 610 receives as input one or more FOV images of a microscope slide. In many embodiments, grading models can also receive as input one or more feature vectors computed previously in association with a RI model. The grading model 610 accesses the trained grading model coefficients 530 and generates pathology scores for each of the input FOV images. Generated pathology scores for the input FOV image may indicate various measures, such as (but not limited to) a probability of a presence of a disease, a severity of a disease, an aggressiveness of a disease, or other metric in a patient associated with the microscope slide, according to one embodiment.

An example of input and output vectors relevant to RI models and grading models are discussed below. In a variety of embodiments, input vectors may include but are not limited to) a set of whole slide images captured by a whole slide scanner of a microscope slide for a RI model and/or feature vectors for whole slide and/or FOV images. In many embodiments, the resulting output vectors of a RI model may include (but are not limited to) ROI coordinates for detected ROIs on the microscope slide and/or corresponding feature detection scores for each of the detected ROIs. Input vectors for a grading model in accordance with numerous embodiments of the invention may include a set of FOV images of the microscope slide and/or feature detection scores generated from inputting the whole slide images of the microscope slide into a RI model. In a number of embodiments, the input set of FOV images are captured automatically by a microscope based upon the detection of ROIs in the whole slide image and generation of ROI coordinates by a RI model. The resulting output vectors of the grading model may include a set of pathology scores for the ROI depicted in the input FOV images. The output pathology scores in accordance with some embodiments of the invention may include a probability of occurrence for various types of diseases in a patient associated with the microscope slide. For example, the output pathology score may include a Gleason score indicating the severity of a prostate cancer in the patient.

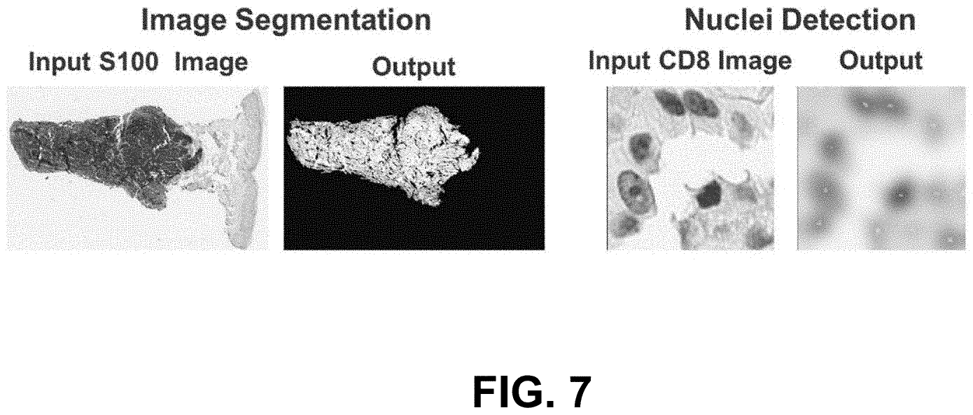

FIG. 7 is an example of an input image and output of a feature detection algorithm used in a RI model and a grading model of a ROI inspection system in accordance with an embodiment of the invention. The feature detection algorithm used in this example is a phase stretch transform. The phase stretch transform is used to perform image segmentation and nuclei detection of cells in an image containing cells.

### Interface

In certain embodiments, detected ROIs, ROI coordinates of each of the detected ROIs, information relating to the feature vectors for each of the detected ROIs, and/or pathology scores for each of the detected ROIs may be stored in a database and/or may be displayed in an augmented reality interface overlaid on whole slide images and/or FOV images. The augmented reality assists a pathologist and/or user of a ROI inspection system in identifying ROIs on a microscope slide and assessing the microscope slides with pathology scores. By overlaying the generated data from the RI model and the grading model, the pathologist may more quickly analyze relevant aspects of a ROI on the microscope slide than if the pathologist is manually inspecting a microscope slide without the relevant information provided in the augmented reality interface.

In many embodiments, each of the pathology scores is displayed on the display of a client device, with the pathology scores displayed concurrently with the whole slide image. In this case, each of the pathology scores may be displayed in a region of the whole slide image corresponding to a detected ROI and overlapping a region where the whole slide image is displayed. In various embodiments, each of the pathology scores is concurrently displayed on the display of the client device with a corresponding FOV image. In this case, each of the pathology scores can be displayed in a region overlapping a region where the corresponding FOV image is displayed. In a further embodiment, a pathologist and/or user of the ROI inspection system may add their own annotations to the whole slide images and/or FOV images. The annotations may be stored and appear overlaid on the whole slide images and/or FOV images in the augmented reality interface. Client devices in accordance with various embodiments of the invention may automatically store and display annotations in the augmented reality interface, for example a tissue grade corresponding to a specimen on the microscope slide.

### Process

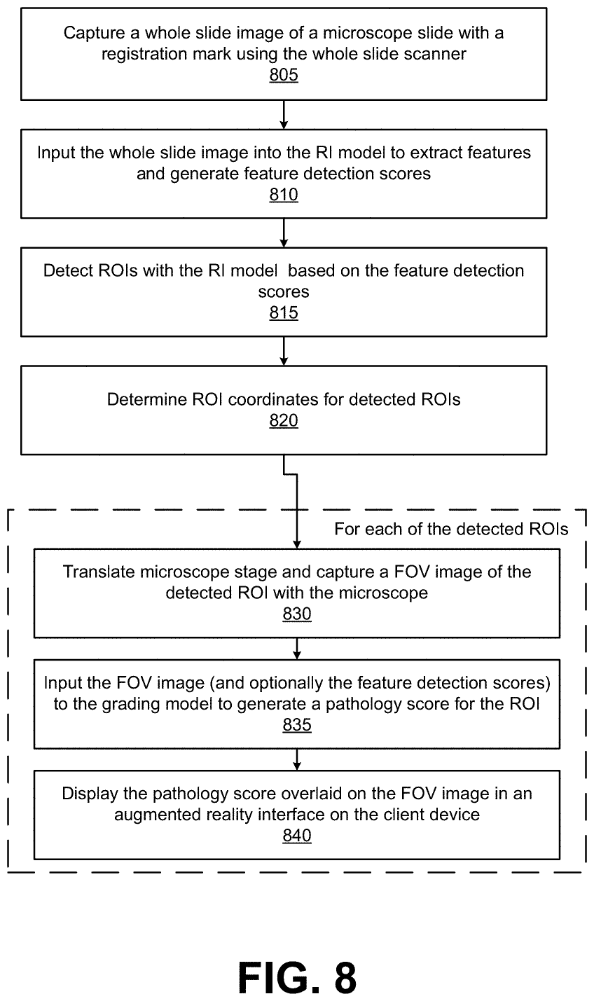

FIG. 8 is a flowchart illustrating a process for detecting ROIs on a microscope slide and providing guidance relating to analyzing the ROI in accordance with an embodiment of the invention. A whole slide image of a microscope slide is captured 805 by a whole slide scanner. The whole slide image is inputted 810 into a RI model to extract features of the whole slide image and/or generate feature vectors. The extracting of features may include first segmenting the whole slide image before extracting features in each of the segments of the whole slide image. The RI model in accordance with some embodiments of the invention may then generate a feature vector for each segment containing one or more extracted features. In a variety of embodiments, feature vectors may be generated from the whole slide image without necessarily segmenting the image. RI models can then be used to detect 815 ROIs on the microscope slide based on the feature vectors and determine 820 coordinates in a coordinates system for each of the detected ROIs relative to the registration marks on the microscope slide.

For each of the detected ROIs, the following steps can be performed, according to one embodiment. In a number of embodiments, the client device can be used to instruct a microscope stage controller to translate 830 the microscope stage to position the detected ROI in the FOV of a microscope. A FOV image of the detected ROI is captured 830 and inputted 835 to a grading model. In some embodiments, the feature vectors generated by the RI model can also be inputted 835 to the grading model. The grading model can be used to generate a pathology score for the detected ROI. The pathology score is displayed 840 simultaneously with the FOV image of the detected ROI in an augmented reality interface on the display of the client device. In one embodiment, for example, the pathology score is displayed 840 overlaid on the FOV image and overlapping a region of the display where the FOV images is displayed.

While specific processes for detecting ROIs are described above, any of a variety of processes can be utilized to detect ROIs as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

### Exemplary Computing Devices

FIG. 9 is a high-level block diagram illustrating physical components of an exemplary computer 900 that may be used as part of a client device, application server, and/or data store in accordance with some embodiments of the invention. Illustrated is a chipset 910 coupled to at least one processor 905. Coupled to the chipset 910 is volatile memory 915, a network adapter 920, an input/output (I/O) device(s) 925, a storage device 930 comprising non-volatile memory, and a display 935. The display 935 may be an embodiment of the display 230 of the client device 140. In one embodiment, the functionality of the chipset 910 is provided by a memory controller 911 and an I/O controller 912. In another embodiment, the memory 915 is coupled directly to the processor 905 instead of the chipset 910. In some embodiments, memory 915 includes high-speed random access memory (RAM), such as DRAM, SRAM, DDR RAM or other random access solid state memory devices.

The storage device 930 is any non-transitory computer-readable storage medium, such as a hard drive, compact disk read-only memory (CD-ROM), DVD, or a solid-state memory device. The memory 915 holds instructions and data used by the processor 905. The I/O device 925 may be a touch input surface (capacitive or otherwise), a mouse, track ball, or other type of pointing device, a keyboard, or another form of input device. The display 935 displays images and other information from the computer 900. The network adapter 920 couples the computer 900 to the network 190.

As is known in the art, a computer 900 can have different and/or other components than those shown in FIG. 9. In addition, the computer 900 can lack certain illustrated components. In one embodiment, a computer 900 acting as a server 150 may lack a dedicated I/O device 925, and/or display 935. Moreover, the storage device 930 can be local and/or remote from the computer 900 (such as embodied within a storage area network (SAN)), and, in one embodiment, the storage device 930 is not a CD-ROM device or a DVD device.

Generally, the exact physical components used in a client device can vary in size, power requirements, and performance from those used in an application server and/or a data store. For example, client devices which will often be home computers, tablet computers, laptop computers, or smart phones, will include relatively small storage capacities and processing power, but will include input devices and displays. These components are suitable for user input of data and receipt, display, and interaction with notifications provided by the application server. In contrast, the application server may include many physically separate, locally networked computers each having a significant amount of processing power for carrying out the analyses described above. In one embodiment, the processing power of the application server is provided by a service such as Amazon Web Services™ or Microsoft Azure™. Also in contrast, the data store may include many physically separate computers each having a significant amount of persistent storage capacity for storing the data associated with the application server.

As is known in the art, the computer 900 is adapted to execute computer program modules for providing functionality described herein. A module can be implemented in hardware, firmware, and/or software. In one embodiment, program modules are stored on the storage device 930, loaded into the memory 915, and executed by the processor 905.

### Benefits

ROI inspection systems in accordance with a number of embodiments of the invention can provide the benefit of an automated system for detecting relevant ROIs on a microscope slide, generating feature detection scores and pathology scores for each of the detected ROIs, and displaying the ROIs, the feature information, and pathology scores in an augmented reality interface for a user of the ROI inspection system. In many embodiments, ROI inspection systems may provide the benefit of greatly increasing the throughput of a pathologist or a pathology laboratory by reducing the amount of time a pathologist is required to manually and visually inspect microscope slides. Additionally, the ROI inspection systems in accordance with several embodiments of the invention may increase the accuracy of assessing pathology microscope slides by notifying a pathologist of a detected ROI on a microscope.

A benefit of ROI inspection systems in accordance with a number of embodiments of the invention can be that the coordinate system for identifying locations of ROIs can be established regardless of whether the whole slide scanner and the microscope capture their respective images at different image resolutions. While it may be difficult to establish a mapping of ROIs on a microscope slide using other methods due to an inconsistency in image resolution between the whole slide scanner and the microscope, the use of the registration marks in establishing spatial references for the coordinate system allows for an effective means of coordinating the workflow between the whole slide scanner and the microscope.

### ADDITIONAL CONSIDERATIONS

It is to be understood that the figures and descriptions of the present disclosure have been simplified to illustrate elements that are relevant for a clear understanding of the present disclosure, while eliminating, for the purpose of clarity, many other elements found in a typical system. Those of ordinary skill in the art may recognize that other elements and/or steps are desirable and/or required in implementing the present disclosure. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

Some portions of above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times to refer to these arrangements of operations as modules, without loss of generality. The described operations and their associated modules may be embodied in software, firmware, hardware, or any combinations thereof.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

While particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope of the ideas described herein.

What is claimed is:

1. A method comprising:
obtaining a whole slide image of a microscope slide comprising a registration mark with a whole slide imaging device, wherein the registration mark is associated with an origin of a coordinate system;
inputting the whole slide image into a region identification (RI) model to extract features of the whole slide image and generate feature vectors for the extracted features;
detecting a presence of a region of interest (ROI) based on the feature vectors;
determining a set of coordinates of the ROI in the coordinate system;
translating a microscope stage of a microscope holding the microscope slide to a position corresponding to the coordinates of the ROI;
capturing a field of view (FOV) image with the microscope, wherein the FOV image includes at least a portion of the ROI;
inputting the FOV image into a grading model to determine a pathology score, the pathology score indicating a likelihood of a presence of a disease; and
displaying the FOV image and the pathology score on a display device.

2. The method of claim 1 further comprising marking the microscope slide with the registration mark, wherein the registration mark comprises an etched pattern in the microscope slide.

3. The method of claim 1, further comprising displaying the whole slide image on the display device.

4. The method of claim 1, wherein the registration mark is configured so as to define the coordinate system as having sub-micron resolution.

5. The method of claim 1, wherein the translating the microscope stage comprises changing a level of magnification of the microscope.

6. The method of claim 1, wherein the displaying the pathology score on the display device further comprises concurrently displaying the pathology score with the whole slide image, wherein the pathology score is displayed in a region corresponding to the ROI and overlapping a region where the whole slide image is displayed.

7. The method of claim 1, wherein the displaying the pathology score further comprises concurrently displaying with the FOV image, wherein the pathology score is displayed in a region overlapping a region where the FOV image is displayed.

8. The method of claim 1, further comprising:
generating an annotation associated with the whole slide image and the FOV image, the annotation comprising any combination of the feature vectors, the pathology score, the coordinates of the ROI, and a text string description inputted by a user; and
storing the whole slide image, the FOV image, and the associated annotation.

9. The method of claim 1, further comprising pre-processing the whole slide image using at least one technique from a group consisting of:
image denoising, contrast enhancement, uniform aspect ratio, rescaling, normalization, segmentation, cropping, object detection, dimensionality deduction/increment, brightness adjustment, and data augmentation techniques, image shifting, flipping, zoom in/out, rotation, and thresholding and morphological operations.

10. The method of claim 1, wherein the RI model comprises:
a set of RI model coefficients trained using a first set of whole slide training images, a second set of whole slide training images, a set of training ROI coordinates, and a function relating one of the whole slide images and the RI model coefficients to the presence of the ROI and the coordinates of the ROI, wherein:
each of the first set of whole slide training images comprises a registration mark and at least one ROI, and
each of the training ROI coordinates corresponds to the at least one ROI of one of the whole slide training images.

11. The method of claim 10, wherein the first set of whole slide training images and the second set of whole slide training images are captured with the whole slide imaging device.

12. The method of claim 1, wherein the grading model comprises:
a set of grading model coefficients trained using a set of features derived from the FOV training images, each of the FOV training images comprising an RI identified by inputting a whole slide training image into the RI model,
a set of training pathology scores each corresponding to one of the whole slide training images, and
a function relating one of the FOV training images and the grading model coefficients to the pathology score.

13. The method of claim 12, wherein the set of FOV training images are captured with the microscope.

14. The method of claim 1, wherein the extracted features comprise at least one of nuclei, lymphocytes, immune checkpoints, and mitosis events.

15. The method of claim 1, wherein the extracted features comprise at least one of scale-invariant feature transform (SIFT) features, speeded-up robust features (SURF), and oriented FAST and BRIEF (ORB) features.

16. The method of claim 1, wherein the RI model is a convolutional neural network.

17. The method of claim 1, wherein the RI model uses a combination of phase stretch transform, phase-stretch adaptive gradient field extractor, Canny edge detection method, and Gabor filter banks to extract the features of the whole slide image and generate the feature vectors.

18. The method of claim 1, wherein the ROI is a region encompassing a single cell.

19. The method of claim 1, wherein the ROI is a region smaller than a single cell.

20. A non-transitory machine readable medium containing processor instructions, where execution of the instructions by a processor causes the processor to perform a process comprising:
obtaining a whole slide image of a microscope slide comprising a registration mark with a whole slide imaging device, wherein the registration mark is associated with an origin of a coordinate system;
inputting the whole slide image into a region identification (RI) model to extract features of the whole slide image and generate feature vectors for the extracted features;
detecting a presence of a region of interest (ROI) based on the feature vectors;
determining a set of coordinates of the ROI in the coordinate system;
translating a microscope stage of a microscope holding the microscope slide to a position corresponding to the coordinates of the ROI;
capturing a field of view (FOV) image with the microscope, wherein the FOV image includes at least a portion of the ROI;
inputting the FOV image into a grading model to determine a pathology score, the pathology score indicating a likelihood of a presence of a disease; and
displaying the FOV image and the pathology score on a display device.
